(19) United States
(12) Patent Application Publication
(10) Pub. No.: US 2023/0287367 A1
(43) Pub. Date: Sep. 14, 2023

(54) Thermobifida fusca cutinase mutant and its soluble expression method

(71) Applicant: Jiangnan University, Wuxi (CN)

(72) Inventors: Jing WU, Wuxi (CN); Lei WANG, Wuxi (CN); Fangjian ZHU, Wuxi (CN); Sheng CHEN, Wuxi (CN)

(21) Appl. No.: 18/063,732

(22) Filed: Dec. 9, 2022

Related U.S. Application Data
(63) Continuation of application No. PCT/CN2020/095350, filed on Jun. 10, 2020.

(51) Int. Cl. C12N 9/18; C12N 9/90; C12N 15/70

(52) U.S. Cl. CPC C12N 9/18; C12Y 301/01074; C12N 9/90; C12Y 503/04001; C12N 15/70; C12N 2800/101

(57) ABSTRACT

The present disclosure discloses a Thermobifida fusca cutinase mutant and a soluble expression method thereof, belonging to the technical field of enzyme engineering. In the present disclosure, the mutant D204C/E253C and disulfide isomerase DsbC of periplasmic proteins are co-expressed in mutant E. coli Origami B (DE3), but the recombinant E. coli Origami B (DE3)/pSCDsbC-D204C/E253C obtained is easily misfolded and forms a large number of inclusion bodies in the expression process, and the ratio of soluble expression is extremely low. The present disclosure further achieves highly soluble expression of the cutinase mutant D204C/E253C by co-expression with molecular chaperonin DsbC in the mutant E. coli Origami B (DE3), and has certain industrial application prospects.

### TECHNICAL FIELD

The present disclosure relates to a Thermobifida fusca cutinase mutant and its soluble expression method, belonging to the technical field of enzyme engineering.

### BACKGROUND

Thermobifida fusca cutinase belongs to the α/β hydrolase family. A β-pleated sheet in the center is surrounded by an α-helix and a loop region, forming a sandwich structure. An active center is composed of a Ser-His-Asp catalytic triangle. T. fusca cutinase is a multifunctional enzyme capable of hydrolyzing macromolecular polyesters, insoluble triglycerides and small molecular soluble esters. Also, T. fusca cutinase may catalyze esterification of multiple acids and alcohols, and transesterification of esters and alcohols. Cutinase is different from lipase in the characteristic that the active center of the cutinase is not covered by a “cap” structure, which catalyzes a key amino acid Ser to expose to solvents. Hence, some cutinase may also hydrolyze high molecular weight polyester PET (polyethylene terephthalate). However, PET is highly crystalline, which reduces the accessibility of the cutinase to the polyester chain in PET, and seriously affects the catalytic efficiency of the cutinase. At a glass transition temperature (69-80° C.), the movement of the molecular chain of PET intensifies and forms many voids, increasing the accessibility to the active center of the enzyme molecule, and achieving a more obviously catalytic effect.

### SUMMARY

The present disclosure provides a cutinase mutant with a high thermal stability. On the cutinase shown in SEQ ID NO. 1, an amino acid of at least one of the following sites is mutated: positions 61, 89, 204, and 253.

In one embodiment, the cutinase mutant is (a) or (b):
(a) on the cutinase shown in SEQ ID NO. 1, amino acids at positions 61 and 89 are mutated into cysteine, and an amino acid sequence of the resulting mutant is shown in SEQ ID NO. 2; and
(b) on the cutinase as shown in SEQ ID NO. 1, glutamic acid at position 204 and aspartic acid at position 253 are mutated into cysteine, and an amino acid sequence of the resulting mutant is shown in SEQ ID NO. 3.

The present disclosure further provides a gene encoding the cutinase mutant.

In one embodiment, the gene encoding the mutant D204C/E253C contains a nucleotide sequence shown in SEQ ID NO. 4.

The present disclosure further provides an expression vector carrying the gene.

In one embodiment, the expression vector is a plasmid of pET series.

In one embodiment, the expression vector is pSCDsbC, of which a nucleotide sequence is shown in SEQ ID NO. 5.

The present disclosure further provides microbial cells expressing the cutinase mutant.

In one embodiment, the microbial cells are Escherichia coli.

In one embodiment, the E. coli is E. coli BL21, E. coli BL21 (DE3), E. coli JM109, E. coli DH5α or E. coli TOP10.

The present disclosure further provides a soluble expression method of the cutinase mutant, comprising: co-expressing the cutinase mutant with disulfide oxidoreductase DsbC of periplasmic proteins.

In one embodiment, an amino acid sequence of the disulfide oxidoreductase DsbC of periplasmic proteins is shown in SEQ ID NO. 6.

In one embodiment, the method comprises: ligating a gene encoding the cutinase mutant and a gene encoding the disulfide oxidoreductase of periplasmic proteins with a vector separately, and transforming the ligated genes and vectors into microbial cells for expression.

In one embodiment, the method further comprises: adding an RBS sequence of a ribosome binding site upstream of genes.

In one embodiment, the method uses a plasmid pSC as an expression vector and E. coli Origami B (DE3) as a host to co-express the cutinase mutant and the disulfide oxidoreductase of periplasmic proteins.

In one embodiment, the nucleotide sequence of the plasmid pSC is shown in SEQ ID NO. 7.

The present disclosure further provides application of the cutinase mutant in the field of chemical industry or textiles.

In one embodiment, the application refers to modification of PET fiber or fabric thereof.

In one embodiment, the modification is to perform an enzymatic hydrolysis reaction on PET fiber with the cutinase mutant at a dose of 10 U/g substrate, a bath ratio of 1:40, and a temperature of 80° C.

In one embodiment, Triton X-100 with a final concentration of 0.5-2% is added to the enzymatic hydrolysis reaction.

Beneficial Effects:

1. In the present disclosure, the glutamic acid at position 204 and the aspartic acid at position 253 near the catalytic triangle are selected and mutated into cysteine to introduce a pair of disulfide bonds to stabilize the catalytic triangle; and amino acids at positions 61 and 89 are mutated into cysteine to form a pair of disulfide bonds to stabilize the N-terminal domain of the cutinase, so that the cutinase mutant has a high thermal stability. The mutant can catalyze hydrolysis of ester bonds of PET fiber under the reaction condition of glass transition temperature (Tg) of PET to achieve the purpose of hydrophilic modification.

2. The present disclosure provides a soluble expression method of a mutant D204C/E253C, aiming at the defects that the mutant D204C/E253C is easily misfolded and forms a large number of inclusion bodies in the expression process due to introduction of disulfide bonds, and the ratio of soluble expression is extremely low. By co-expressing the mutant D204C/E253C and the disulfide oxidoreductase DsbC of periplasmic proteins in the mutant E. coli Origami B (DE3), abnormal disulfide bonds are isomerized, and a highly soluble expression is achieved.

### BRIEF DESCRIPTION OF FIGURES

FIG. 1A shows an SDS-PAGE diagram of different proteins, where M: Protein molecular weight standard; Lanes 1-3 in (A): Extracellular supernatant, wall broken supernatant and wall broken precipitate of recombinant E. coli BL21 (DE3)/pET-20b(+)-cut, respectively; Lanes 4-6: Extracellular supernatant, wall broken supernatant and wall broken precipitate of recombinant E. coli BL21 (DE3)/pET-20b(+)-T61C/T89C, respectively.

Lanes 1-3 in FIG. 1B: Extracellular supernatant, wall broken supernatant and wall broken precipitate of recombinant E. coli BL21 (DE3)/pET-20b(+)-D204C/E253C, respectively.

FIG. 2 shows an SDS-PAGE diagram of purified wild-type cutinase and mutants, where M: Protein molecular weight standard; Lane 2: Wild-type T. fusca cutinase; Lanes 3-4: Mutant T61C/T89C; Lanes 5-6: Mutant D204C/E253C.

FIG. 3 shows enzymatic activities of cutinase and mutants at different temperatures, where WT: Wild-type cutinase.

FIG. 4A shows a thermal stability of the mutant D204C/E253C at 80° C.

FIG. 4B shows a thermal stability of the mutant D204C/E253C at 90° C.

FIG. 5 shows light absorption values of treatment residues of the wild-type cutinase and the mutant D204C/E253C at 240 nm.

FIG. 6 shows a difference in wettability of PET fiber treated with different cutinase.

FIG. 7A shows an SEM photo of PET fiber treated with a buffer at 50° C.

FIG. 7B shows an SEM photo of PET fiber treated with the wild-type cutinase at 50° C.

FIG. 7C shows an SEM photo of PET fiber treated with a buffer at 80° C.

FIG. 7D shows an SEM photo of PET fiber treated with the mutant D204C/E253C at 80° C.

FIG. 8 shows a construction process of a recombinant plasmid pSCDsbC-D204C/E253C.

FIG. 9 shows a double digestion verification of the recombinant plasmid pSCDsbC-D204C/E253C, where M: 5000 bp DNA marker; 1: Sample.

FIG. 10 shows expression of recombinant E. coli BL21(DE3)/pET20b-D204C/E253C and E. coli Origami B (DE3)/pSCDsbC-D204C/E253C; (A): Expression of pET20b-D204C/E253C in host E. coli BL21(DE3); (B): Expression of pSCDsbC-D204C/E253C in host E. coli Origami B (DE3); M: Protein Marker molecular weight standard (10-200 kDa); Lanes 1, 2 and 3: Extracellular supernatant, wall broken supernatant and wall broken precipitate, respectively.

### DETAILED DESCRIPTION

Enzymatic activity determination method: The cutinase activity is determined by continuous spectrophotometry. A reaction system (1.5 mL) includes 30 μL of an appropriately diluted enzyme solution and 1470 μL of a Tris-HCl buffer (10 mmol·L⁻¹, pH 8.0) containing sulfur sodium deoxycholate (50 mmol·L⁻¹) and pNPB (50 mmol·L⁻¹). A production rate of p-nitrophenol is recorded at 405 nm.

Definition of enzymatic activity: At 37° C., the amount of enzyme required to hydrolyze p-nitrophenyl butyrate to produce 1 μmol of p-nitrophenol per minute is an enzymatic activity unit.

Thermal stability analysis method: An enzyme solution is diluted appropriately with a Tris-HCl buffer (10 mmol·L⁻¹, pH 8.0), and heat is preserved at 90° C. for 10 min. The enzymatic activity of the enzyme solution before and after the heat preservation is accurately measured, respectively. In an analysis process, the cutinase activity before heat preservation is expressed as 100%, and is used as a standard for calculating the residual enzymatic activity of the cutinase after the heat preservation to determine the thermal stability of the cutinase at 90° C.

Example 1 Preparation of Mutant Enzymes and Wild-Type Enzyme

(1) Site-directed mutation: Based on a cutinase gene of Thermobifida fusca (NCBI database registration number: AAZ54921.1), using plasmids pET20b(+)-cut and pET24a(+)-cut carrying the cutinase gene as templates (the plasmids were disclosed in the paper Gene Identification, High-efficiency Expression and Molecular Modification of Thermobifida fusca cutinase), primers were respectively designed and synthesized for conducting site-directed mutation on cutinase genes by a rapid PCR (the underlined were mutant bases).

(a) The plasmid pET20b/cut carrying the cutinase gene was used as a template:

Site-directed mutagenic primers introduced into a sequence T61C for mutation were as follows:

Forward primer:
(SEQ ID NO. 8)
GCGGTGGCGATCTCCCCCGGCTACTGTGGCACTGAGGCT
Reverse primer:
(SEQ ID NO. 9)
CCAGGCGATGGAAGCCTCAGTGCCACAGTAGCCGGGGGA

The site-directed mutagenic primers introduced into a sequence T89C for mutation were as follows:

Forward primer:
(SEQ ID NO. 10)
GTCATCACCATCGACACCATCACCTGTCTCGACCAGCCG
Reverse primer:
(SEQ ID NO. 11)
TGCCCGGCTGTCCGGCTGGTCGAGACAGGTGATGGTGTC

The site-directed mutagenic primers introduced into a sequence D204C for mutation were as follows:

Forward primer:
(SEQ ID NO. 12)
ATCAGCAAGGCCTACCTGGAGCTGTGTGGCGCAACCCAC
Reverse primer:
(SEQ ID NO. 13)
GTTCGGGGCGAAGTGGGTTGCGCCACACAGCTCCAGGTA

The site-directed mutagenic primers introduced into a sequence E253C for mutation were as follows:

Forward primer:
(SEQ ID NO. 14)
CGCGACGGACTCTTCGGCGAGGTCTGTGAGTACCGCTCC
Reverse primer:
(SEQ ID NO. 15)
GAACGGGCAGGTGGAGCGGTACTCACAGACCTCGCCGAA

(b) A plasmid pET24a/cut carrying the cutinase gene was used as a template:

The site-directed mutagenic primers introduced into the sequence D204C for mutation were as follows:

Forward primer:
(SEQ ID NO. 16)
GCCTATCTGGAACTGTGTGGTGCCACCCATTTTGCCCCG
Reverse primer:
(SEQ ID NO. 17)
CGGGGCAAAATGGGTGGCACCACACAGTTCCAGATAGGC

The site-directed mutagenic primers introduced into the sequence E253C for mutation were as follows:

Forward primer:
(SEQ ID NO. 18)
CTGTTCGGCGAAGTGTGTGAATACCGCAGC
Reverse primer:
(SEQ ID NO. 19)
GCTGCGGTATTCACACACTTCGCCGAACAG

All PCR reaction systems included: 10 μl of 5×PS buffer, 4 μL of dNTPs Mix (2.5 mM), 1 μL of forward primer (10 μM), 1 μL of reverse primer (10 μM), 1 μL of template DNA, and 0.5 μL. of Prime STAR HS DNA polymerase (5 U/μL), supplemented with double distilled water to 50 μL. All PCR amplification conditions were: pre-denaturation at 94° C. for 4 min; 30 cycles (98° C. for 10 s, 58° C. for 5 s, 72° C. for 6 min) carried out subsequently; and extension continued at 72° C. for 10 min. PCR products were digested by Dpn I (Fermentas) and transformed into competent cells of E. coli JM109. The competent cells were cultured overnight in an LB solid medium (containing 100 μg/mL ampicillin/kanamycin). Then, single clones were picked and cultured in an LB liquid medium (containing 100 μg/mL of ampicillin/kanamycin), and plasmids were extracted and sequenced.

(2) Expression and purification of mutant enzymes and the wild-type enzyme: The plasmid sequenced as correct mutation (a template plasmid was directly used for the wild-type enzyme) was transformed into competent cells of an expression host E. coli BL21 (DE3). Single clones were picked and transformed into the expression host E. coli BL21 (DE3) and grown in the LB liquid medium (containing 100 μg/mL ampicillin) for 8-10 h. A seed fermentation broth was transferred into the TB liquid medium (containing 100 μg/mL ampicillin) at an inoculum concentration of 4%. The E. coli was incubated on a shaker at 37° C. until OD₆₀₀=0.6-0.8. IPTG with a final concentration of 0.01 mM was added to induce extracellular expression, and the cells were incubated and fermented on a shaker at 25° C. for 48 h. The fermentation broth was centrifuged at 4° C. and 10000 rpm for 15 min to remove the cells, and the supernatant was collected.

70% (w/v) solid ammonium sulfate was added to the supernatant obtained by the above method. The supernatant was salted out at 4° C. overnight and centrifuged at 10,000 rpm for 20 min. The precipitate was added and dissolved in an appropriate amount of buffer A (Tris-HCl, 20 mmol/L, pH 8.0), and dialyzed in the buffer A overnight. The dialyzed sample was centrifuged at 12,000 rpm for 20 min, and then filtered through a 0.45 μm membrane to prepare a loading sample. A DEAE-Sepharose FF anion exchange column was pre-balanced with the buffer A and then loaded with the sample. After unconjugated components were washed off with the buffer A, linear gradient elution was conducted using a mixed solution of buffer A and buffer B (buffer A containing 1 M NaCl). The full-range flow rate was 1 mL/min, and the detection wavelength was 280 nm. The eluent with an absorption peak was collected for detecting cutinase activity and protein electrophoresis. The enzymatic activity components were then separated and purified by a monoQ pre-loaded anion exchange column under the above similar conditions, and a purified product was finally obtained through detection of the cutinase activity and the protein electrophoresis (as shown in FIG. 2).

Example 2 Analysis on Thermal Stability of Wild-Type Cutinase and Mutants Thereof

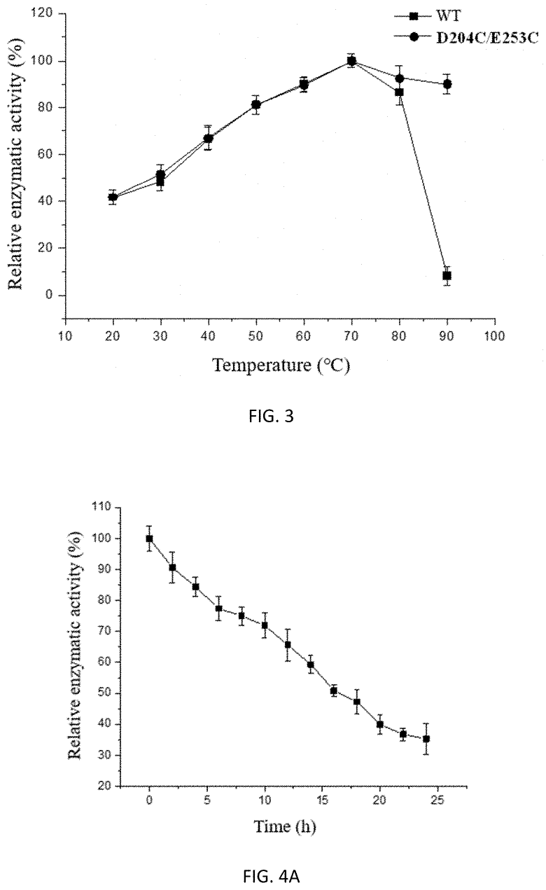

The Tris-HCl buffer (10 mmol/L, pH 8.0) was used for measuring the cutinase activity in a range of 20-90° C. at an interval of 10° C. to determine the optimal temperature of the enzyme. The highest enzymatic activity was counted as 100%, and the relative enzymatic activity at each temperature was calculated. As shown in FIG. 3, at 70° C., the activities of the wild-type enzyme and mutant enzymes were the highest, reaching 56 U·mL⁻¹ and 9 U·mL⁻¹ respectively; and at 90° C., the relative activity of the mutant enzymes was 92.2% and that of the wild-type enzyme was 8.7%.

In the Tris-HCl buffer (10 mmol/L, pH 8.0), the mutant D204C/E253C was kept at 80° C. and 90° C. respectively, and periodically sampled to measure the residual enzymatic activity to determine the thermal stability of the enzyme. The results showed that the wild-type cutinase was inactivated after heat preservation for 10 min at 80° C. and 90° C., and had a residual enzymatic activity of 10.7% after heat preservation at 70° C. for 10 min.

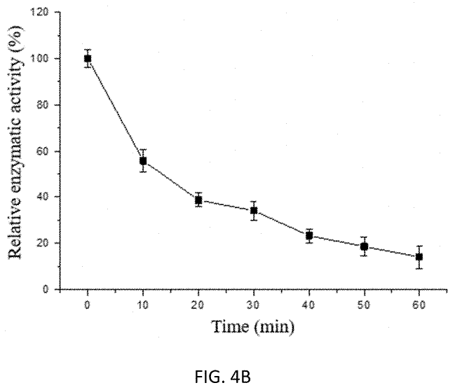

The mutant D204C/E253C had a half-life period of 16 h at 80° C., and still had an activity of 55.6% even after heat preservation at 90° C. for 10 min (FIG. 4), indicating that the mutant D204C/E253C had a good thermal stability at 80° C. and 90° C.

Example 3 Application of Cutinase to Modification of PET Fiber

(1) Enzymatic modification: 1 g of polyester fabric (PET fiber) was washed in a water bath at 60° C. for 30 min, and then put into a phosphate buffer (pH 7.0) at a bath ratio of 1:40. The supernatant of a fermentation broth of the cutinase or the cutinase mutant prepared in Example 1 with an enzyme content of 400 U was added to the above treatment solution, such that the final enzyme concentration of the cutinase is 10 U/mL. Optionally, a fiber penetrant Triton X-100 with a final concentration of 1% was also added to the enzymatic hydrolysis system. The whole system was sealed and treated in a water bath thermostatic oscillator for 24 h. After the treatment, the fabric was taken out and washed thoroughly with distilled water at 60° C.

(2) UV absorbance test of hydrolysates of polyester and PET film: Treatment residues of the wild cutinase and mutants were diluted 10 times. The UV absorbance value of the cutinase treatment residues was measured by a UV spectrophotometer at 240 nm for different treatment time. A blank sample was the cutinase treatment solution not added to polyester fiber under the same conditions.

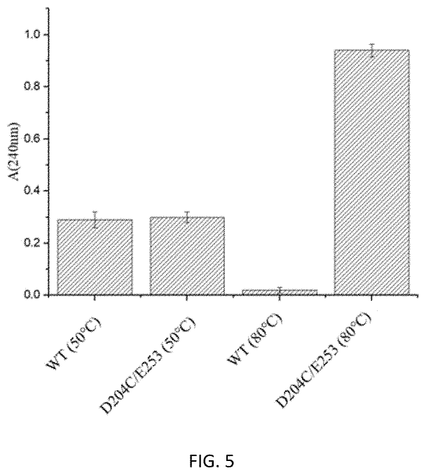

At 80° C., a light absorption value of the treatment residue of the mutant D204C-E253C at 240 nm was 3.13 times that of the mutant D204C-E253C at 50° C. and 3.24 times that of the wild-type cutinase at 50° C. (FIG. 5).

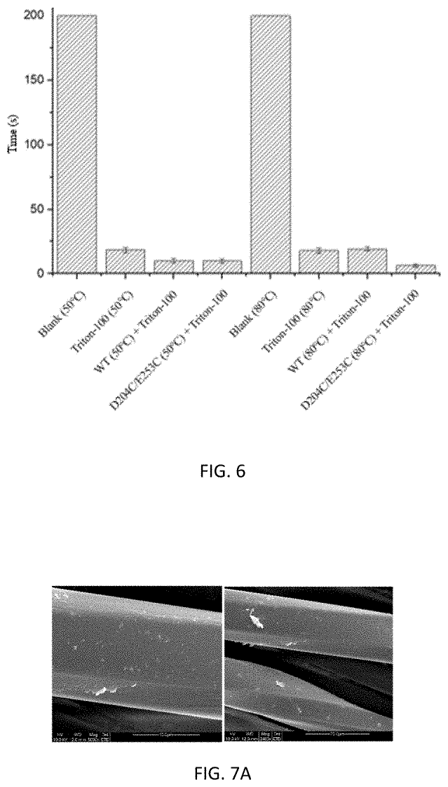

(3) Wettability: The cutinase treated PET fiber was cleaned and put in an oven for drying. The dried PET fiber was spread, and then the PET fiber was balanced at 25° C. and a relative humidity of 65% for 24 h. 20 μl of deionized water was pipetted and dropped onto the same PET fiber continuously in 9 different areas. During this process, the duration from the time when the water drops just touched the PET fiber to the time when the water drops were completely absorbed into the PET fiber was recorded, and then the data were averaged. As shown in FIG. 6, in the presence of Triton X-100, the wettability of the PET fiber treated with the mutant D204C/E253C was 1.62 times higher than that treated with the wild-type T. fusca cutinase.

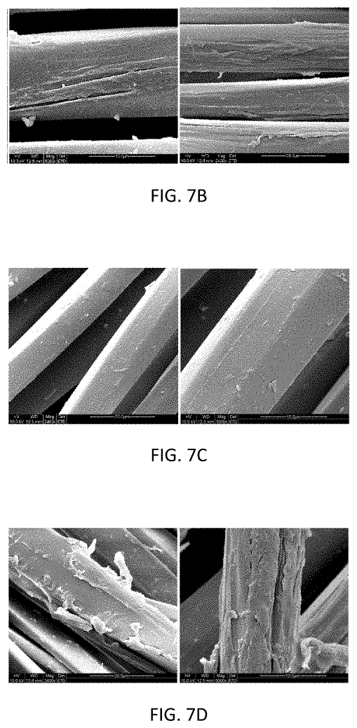

(4) SEM analysis of change of surface micromorphology of PET fiber caused by enzyme treatment: The surface micromorphology of PET fiber was changed by the enzyme treatment. PET fiber after the enzyme treatment was photographed by SEM, and the fiber surface morphology was shown in FIG. 7. FIG. 7A is an SEM photo of PET fiber treated with a buffer at 50° C., from which it can be seen that the PET fiber surface is clear and smooth. FIG. 7B is an SEM photo of PET fiber treated with the wild-type cutinase at 50° C., from which it can be seen that there are obvious etch marks on the fiber surface. FIG. 7C is an SEM photo of PET fiber treated with a buffer at 80° C., from which it can be seen that the PET fiber surface is clear and smooth. FIG. 7D is an SEM photo of PET fiber treated with the mutant D204C/E253C at 80° C., from which it can be seen that there are obvious etch marks on the fiber surface, and the etch marks (also more obvious) on the PET fiber surface treated with the mutant D204C/E253C are more than those treated with the wild-type cutinase, indicating that the mutant D204C/E253C is more effective than the wild-type cutinase in modifying the PET fiber.

Example 4 Construction and Highly Soluble Expression of Recombinant E. coli Origami B (DE3)/pSCDsbC-D204C/E253C

(1) Construction of recombinant E. coli Origami B (DE3)/pSCDsbC-D204C/E253C

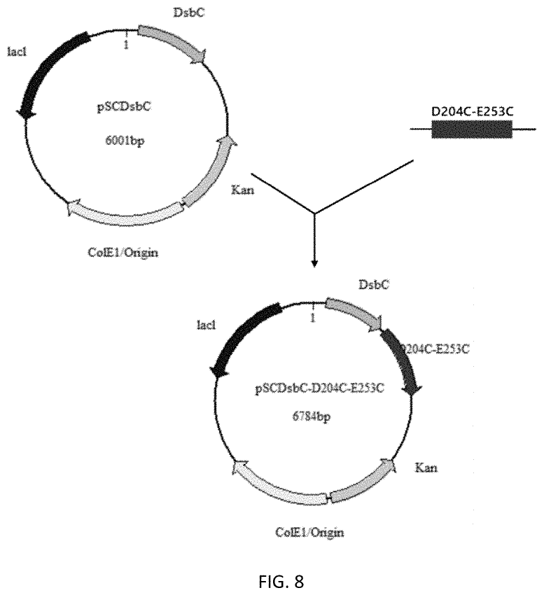

A plasmid pET-20b(+)-D204C/E253C was double digested, and gel extraction was conducted. The extracted mutant D204C/E253C genes were respectively ligated to a cloning vector pSCDsbC at 16° C., and then transformed into E. coli JM109. After overnight culture on a plate, single colonies were picked and cultured for about 10 h. The two recombinant plasmids were extracted for conducting digestion verification respectively. The specific construction process is shown in FIG. 8.

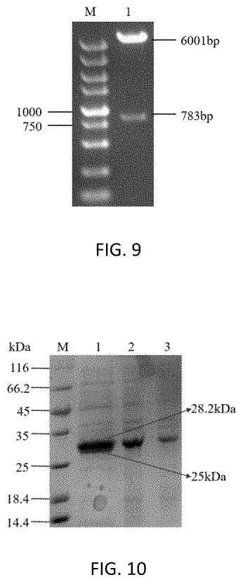

By digestion verification, two bands appeared in electrophoresis, where one at about 783 bp was the mutant D204C/E253C gene, and the other at about 6001 bp was the pSCDsbC vector (FIG. 9). The plasmid with correct digestion verification was sent for sequencing, and the sequencing result was correct. Hence, the construction was successful.

(2) Expression of recombinant E. coli Origami B (DE3)/pSCDsbC-D204C/E253C

The plasmid pSCDsbC-D204C/E253C was extracted. The plasmid pSCDsbC-D204C/E253C was transformed into competent cells of E. coli Origami B (DE3), and then the competent cells were spread on an ampicillin resistant LB plate. Single colonies were picked and cultured in 10 mL of the LB liquid medium for 8-10 h, and then the cells were transferred into 50 mL of a ampicillin resistant TB medium at an inoculum concentration of 5%. The cells were cultured at 37° C. until OD₆₀₀=1.5, and then IPTG with a final concentration of 0.4 mmol·L⁻¹ was added and cooled to 25° C. to induce expression for 24 h. The results of SDS-PAGE identification are shown in FIG. 10.

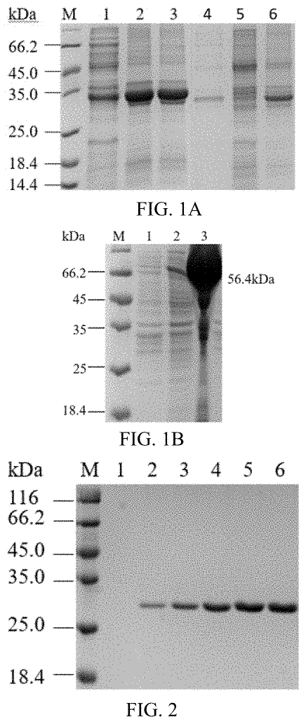

Through electrophoresis analysis, compared with the expression of the wild-type cutinase pET20b-cut in the host E. coli BL21 (DE3) (FIG. 1 (A)), the mutant pET20b-D204C/E253C formed a large number of inclusion bodies (FIG. 1B) when expressed in the host E. coli BL21 (DE3). This is because abnormal folding of the mutant D204C/E253C led to formation of intermolecular disulfide bonds, and the mutant largely gathered in a form of dimers to form the inclusion bodies. In addition, the expression of the mutant pET20b-D204C/E253C in the host E. coli Origami B (DE3) was consistent with that in the host E. coli BL21 (DE3). The soluble expression of the mutant pSCDsbC-D204C/E253C in the host E. coli Origami B (DE3) was significantly increased (FIG. 10), where there were two obvious bands in 25-35 kD in lane 1, which were the cutinase mutant D204C/E253C with a molecular weight of 28.2 kD and DsbC with a molecular weight of 25 kD, respectively. The presence of the DsbC may isomerize intermolecular disulfide bonds formed by wrong oxidation. The results showed that by co-expressing the mutant D204C/E253C and molecular chaperonin DsbC in the mutant E. coli Origami B (DE3), the mutant D204C/E253C was folded and expressed correctly.

The recombinant E. coli Origami B (DE3)/pSCDsbC-D204C/E253C was fermented in the TB medium at 25° C. for 24 h, the cutinase mutant in the fermentation broth was collected, and the thermal stability of an extracellular enzyme obtained by expression was determined according to the method in Example 3. The results are shown in Table 1.

TABLE 1
Extracellular enzymatic activity after 24 h of shaking flask
fermentation at 25° C. and thermal stability at 90°
C. of recombinant E. coli Origami B (DE3)/pSCDsbC-D204C/E253C

| | | | Residual |
|---|---|---|---|
| | 0 min | 10 min | enzymatic |
| Recombinant strain | (U · mL⁻¹) | (U · mL⁻¹) | activity (%) |
| E. coli BL21 | 9 ± 0.5 | 5 ± 0.4 | 55.6 |
| (DE3)/pET-20b(+)- | | | |
| D204C/E253C | | | |
| E. coli Origami B | 61 ± 1.1 | 35 ± 0.9 | 57.4 |
| (DE3)/pSCDsbC- | | | |
| D204C/E253C | | | |

By comparing the enzymatic activity of the wild-type T. fusca cutinase in E. coli BL21 (DE3) and the enzymatic activity of the mutant D204C/E253C in two expression systems (E. coli BL21 (DE3), and E. coli Origami B (DE3)) respectively (Table 1), the E. coli Origami B (DE3)/pSCDsbC-D204C/E253C had an enzymatic activity 6.8 times that of the E. coli BL21 (DE3)/pET20b-D204C/E253C, and a thermal stability at 90° C. basically the same as that of the E. coli BL21 (DE3)/pET20b-D204C/E253C.

### Comparative Example: Construction of Cutinase Mutants at Different Sites

According to the same strategy in Example 1, different sites were mutated respectively, and the mutated cutinase was expressed according to the method in Example 1. The primers were as follows:

TABLE 2
Different mutant sites and primers

| Mutant | | |
|---|---|---|
| site | Primer | Nucleotide sequence |
| Y4C | F: GCCAACCCCTGTGAGCGCGGCCCC | SEQ ID NO. 20 |
| | R: GGGGCCGCGCTCACAGGGGTTGGC | SEQ ID NO. 21 |
| D231C | F: TCAAGCGGTTCGTCTGTAACGACACCCGC | SEQ ID NO. 22 |
| | R: TAGCGGGTGTCGTTACAGACGAACCGCTT | SEQ ID NO. 23 |
| R6C | F: AACCCCTACGAGTGTGGCCCCAACCCGACC | SEQ ID NO. 24 |
| | R: CGGGTTGGGGCCACACTCGTAGGGGTTGGC | SEQ ID NO. 25 |
| G78C | F: GCATCGCCTCCCACTGTTTCGTCGTCATC | SEQ ID NO. 26 |
| | R: TGATGACGACGAAACAGTGGGAGGCGAT | SEQ ID NO. 27 |
| E16C | F: CGACGCCCTGCTCTGTGCCAGCAGCGGC | SEQ ID NO. 28 |
| | R: GGCCGCTGCTGGCACAGAGCAGGGCGTC | SEQ ID NO. 29 |
| K216C | F: GAACATCCCCAACTGTATCATCGGCAAG | SEQ ID NO. 30 |
| | R: ACTTGCCGATGATACAGTTGGGGATGTT | SEQ ID NO. 31 |
| F37C | F: AGCGCCAGCGGCTGTGGCGGCGGCAC | SEQ ID NO. 32 |
| | R: GGTGCCGCCGCCACAGCCGCTGGCGCT | SEQ ID NO. 33 |
| Q99C | F: CAGCCGGGCAGAGTGTCTCAACGCCGCG | SEQ ID NO. 34 |
| | R: GCGCGGCGTTGAGACACTCTGCCCGGCT | SEQ ID NO. 35 |
| G39C | F: CCAGCGGCTTCGGCTGTGGCACCATCTACT | SEQ ID NO. 36 |
| | R: AGTAGATGGTGCCACAGCCGAAGCCGCTGG | SEQ ID NO. 37 |
| A102C | F: GAGCAGCTCAACTGTGCGCTGAACCACATG | SEQ ID NO. 38 |
| | R: CATGTGGTTCAGCGCACAGTTGAGCTGCTC | SEQ ID NO. 39 |
| K147C | F: CGTCCCGACCTGTGTGCCGCCATCCCG | SEQ ID NO. 40 |
| | R: GGGATGGCGGCACACAGGTCGGGACG | SEQ ID NO. 41 |
| V230C | F: GGCTCAAGCGGTTCTGTGACAACGACACCC | SEQ ID NO. 42 |
| | R: GGGTGTCGTTGTCACAGAACCGCTTGAGCC | SEQ ID NO. 43 |
| L152C | F: GCCGCCATCCCGTGTACCCCGTGGCAC | SEQ ID NO. 44 |
| | R: GTGCCACGGGGTACACGGGATGGCGGC | SEQ ID NO. 45 |
| P211C | F: CCCACTTCGCCTGTAACATCCCCAAC | SEQ ID NO. 46 |
| | R: GTTGGGGATGTTACAGGCGAAGTGGGT | SEQ ID NO. 47 |
| P167C | F: GCAGCGTCACCGTGTGTACGCTGATCATC | SEQ ID NO. 48 |
| | R: CGATGATCAGCGTACACACGGTGACGCT | SEQ ID NO. 49 |
| S197C | F: GCCGAGCTCCATCTGTAAGGCCTACCTG | SEQ ID NO. 50 |
| | R: CCAGGTAGGCCTTACAGATGGAGCTCGG | SEQ ID NO. 51 |
| A173C | F: CATCGGGTGTGACCTCGACACGATCGCG | SEQ ID NO. 52 |
| | R: CGAGGTCACACCCGATGATCAGCGTCGG | SEQ ID NO. 53 |
| A210C | F: GCGCAACCCACTTCTGTCCGAACATCC | SEQ ID NO. 54 |
| | R: GGGGATGTTCGGACAGAAGTGGGTTG | SEQ ID NO. 55 |

The enzymatic activity was measured after the cells were broken, and the residual enzymatic activity after heat preservation at 70° C. for 10 min was measured. The results are shown in Table 3.

TABLE 3
Enzymatic activity and thermal stability
of different mutant enzymes

| | | | Residual |
|---|---|---|---|
| | 0 min | 10 min | enzymatic |
| Enzyme | (U · mL⁻¹) | (U · mL⁻¹) | activity (%) |
| T. fusca cutinase | 56 ± 0.5 | 6 ± 0.4 | 10.7 |
| Y4C/D231C | 4 ± 0.8 | /^{a} | /^{b} |
| R6C/G78C | 2 ± 0.7 | /^{a} | /^{b} |
| E16C/K216C | 12 ± 1.0 | 1 ± 0.1 | 8.3 |
| F37C/Q99C | 1 ± 0.3 | /^{a} | /^{b} |
| G36C/A102C | 33 ± 0.3 | 5 ± 0.5 | 15.2 |
| T61C/T89C | 2 ± 0.2 | 1 ± 0.2 | 50 |
| K147C/V230C | 59 ± 0.6 | 4 ± 0.2 | 6.8 |
| L152C/P211C | 47 ± 1.1 | 4 ± 0.3 | 8.5 |
| P167C/S197C | 12 ± 0.6 | 1 ± 0.2 | 8.3 |
| A173C/A210C | /^{a} | /^{a} | /^{b} |
| D204C/E253C | 9 ± 0.5 | 9 ± 0.4 | 100 |

Note:
^{a}No activity of the mutant to pNPB was detected;
^{b}No residual enzymatic activity.

Although the present disclosure has been disclosed as above in preferred examples, it is not intended to limit the present disclosure. Those skilled in the art can make various changes and modifications without departing from the spirit and scope of the present disclosure.

Therefore, the protection scope of the present disclosure should be defined by the claims.

1. A cutinase mutant with high thermal stability, wherein the cutinase mutant is:
(a) on the cutinase shown in SEQ ID NO. 1, amino acids at positions 61 and 89 are mutated into cysteine, and an amino acid sequence of the resulting mutant is shown in SEQ ID NO. 2; or
(b) on the cutinase as shown in SEQ ID NO. 1, glutamic acid at position 204 and aspartic acid at position 253 are mutated into cysteine, and an amino acid sequence of the resulting mutant is shown in SEQ ID NO. 3.

2. The cutinase mutant according to claim 1, wherein an amino acid sequence of the cutinase mutant is shown in SEQ ID NO. 3.

3. A polynucleotide sequence, containing a gene encoding the cutinase mutant according to claim 1.

4. The polynucleotide sequence according to claim 3, wherein the polynucleotide sequence is shown in SEQ ID NO. 4.

5. The polynucleotide sequence according to claim 3, wherein the polynucleotide sequence is an expression vector of the gene shown in SEQ ID NO. 4.

6. The polynucleotide sequence according to claim 5, wherein the expression vector is a plasmid of pET series.

7. The polynucleotide sequence according to claim 5, wherein the expression vector is pSCDsbC, of which a nucleotide sequence is shown in SEQ ID NO. 5.

8. A soluble expression method of the cutinase mutant according to claim 1, comprising: co-expressing the cutinase mutant with disulfide oxidoreductase DsbC of periplasmic proteins.

9. The method according to claim 8, wherein an amino acid sequence of the disulfide oxidoreductase DsbC of periplasmic proteins is shown in SEQ ID NO. 6.

10. The method according to claim 9, wherein the method comprises: ligating a gene encoding the cutinase mutant and a gene encoding the disulfide oxidoreductase of periplasmic proteins with a vector separately, and transforming the ligated genes and vectors into microbial cells for expression.

11. The method according to claim 10, wherein the microbial cells are Escherichia coli (E. coli).

12. The method according to claim 9, wherein the E. coli is E. coli BL21, E. coli BL21 (DE3), E. coli JM109, E. coli DH5a or E. coli TOP10.

13. The method according to claim 12, wherein the method further comprises: adding an RBS sequence of a ribosome binding site upstream of genes.

14. The method according to claim 12, wherein the method uses a plasmid pSC as an expression vector and E. coli Origami B (DE3) as a host to co-express the cutinase mutant and the disulfide oxidoreductase of periplasmic proteins.


SEQUENCE LISTING (55 sequences)

SEQ ID NO: 1 - protein, 261 aa, Thermobifida fusca
ANPYERGPNPTDALLEASSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGLFGEVEEYRSTCPF

SEQ ID NO: 2 - protein, 261 aa, synthetic construct
  REGION 1..261: note = Protein translated from synthetic DNA
ANPYERGPNPTDALLEASSGPFSVSEENVSRLSASGFGGGTIYYPRENNTYGAVAISPGYCGTEASIAWLGERIASHGFVVITIDTITCLDQPDSRAEQLNAALNHMINRASSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGLFGEVEEYRSTCPF

SEQ ID NO: 3 - protein, 261 aa, synthetic construct
  REGION 1..261: note = Protein translated from synthetic DNA
ANPYERGPNPTDALLEASSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLAVMGHSMGGGGTLRLASQRPDLKAAIPLTPWHLNKNWSSVTVPTLIIGADLDTIAPVATHAKPFYNSLPSSISKAYLELCGATHFAPNIPNKIIGKYSVAWLKRFVDNDTRYTQFLCPGPRDGLFGEVCEYRSTCPF

SEQ ID NO: 4 - DNA, 786 bp, synthetic construct
  misc_feature 1..786: note = Synthetic DNA
  source 1..786: mol_type = other DNA
gccaacccttatgagcgtggcccgaaccctaccgatgccctgctggaagcaagcagcggtccgttcagcgtgagcgaggaaaatgtgagccgcttaagtgccagcggctttggcggtggcaccatttactatccgcgcgagaataacacctatggtgccgtggcaattagcccgggctataccggcaccgaagcaagcattgcatggctgggtgaacgcatcgcaagtcatggcttcgtggtgatcaccatcgataccatcaccaccctggatcagccggatagccgtgcagaacagctgaacgccgccctgaatcacatgattaatcgtgccagcagcaccgtgcgtagccgcattgacagtagccgcctggccgtgatgggccatagtatgggtggtggcggtaccctgcgcttagccagccaacgccctgatctgaaagccgcaatcccgctgaccccgtggcatctgaacaaaaactggagcagcgtgaccgtgccgaccctgattattggcgccgatctggatacaattgccccggttgccacccacgccaaacctttctacaatagcctgccgagcagcattagcaaggcctatctggaactgtgtggtgccacccattttgccccgaatatcccgaacaagattattggcaaatatagcgtggcctggctgaagcgctttgtggacaacgacacccgctacacccagtttctgtgccctggccctcgtgatggtctgttcggcgaagtgtgtgaataccgcagcacctgcccgttttaa

SEQ ID NO: 5 - DNA, 6001 bp, synthetic construct
  misc_feature 1..6001: note = Synthetic DNA
  source 1..6001: mol_type = other DNA
agatctcgatcccgcgaaattaatacgactcactataggggaattgtgagcggataacaattcccctctagaaataattttgtttaactttaagaaggagatataccatgaagaaaggttttatgttgtttactttgttagcggcgttttcaggctttgctcaggctgatgacgcggcaattcaacaaacgttagccaaaatgggcatcaaaagcagcgatattcagcccgcgcctgtagctggcatgaagacagttctgactaacagcggcgtgttgtacatcaccgatgatggtaaacatatcattcaggggccaatgtatgacgttagtggcacggctccggtcaatgtcaccaataagatgctgttaaagcagttgaatgcgcttgaaaaagagatgatcgtttataaagcgccgcaggaaaaacacgtcatcaccgtgtttactgatattacctgtggttactgccacaaactgcatgagcaaatggcagactacaacgcgctggggatcaccgtgcgttatcttgctttcccgcgccaggggctggacagcgatgcagagaaagaaatgaaagctatctggtgtgcgaaagataaaaacaaagcgtttgatgatgtgatggcaggtaaaagcgtcgcaccagccagttgcgacgtggatattgccgaccattacgcacttggcgtccagcttggcgttagcggtactccggcagttgtgctgagcaatggcacacttgttccgggttaccagccgccgaaagagatgaaagaattcctcgacgaacaccaaaaaatgaccagcggtaaagtcgacggtaccgagctcatgtacccgcgcggtaacctcgagcatatggaattcggatccgtcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 6 - protein, 236 aa, synthetic construct
  REGION 1..236: note = Protein translated from synthetic DNA
MKKGFMLFTLLAAFSGFAQADDAAIQQTLAKMGIKSSDIQPAPVAGMKTVLTNSGVLYITDDGKHIIQGPMYDVSGTAPVNVTNKMLLKQLNALEKEMIVYKAPQEKHVITVFTDITCGYCHKLHEQMADYNALGITVRYLAFPRQGLDSDAEKEMKAIWCAKDKNKAFDDVMAGKSVAPASCDVDIADHYALGVQLGVSGTPAVVLSNGTLVPGYQPPKEMKEFLDEHQKMTSGK

SEQ ID NO: 7 - DNA, 5270 bp, synthetic construct
  misc_feature 1..5270: note = Synthetic DNA
  source 1..5270: mol_type = other DNA
agatctcgatcccgcgaaattaatacgactcactataggggaattgtgagcggataacaattcccctctagaaataattataccgtcgacggtaccgagctcatgtacccgcgcggtaacctcgagcatatggaattcggatccgtcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 8 - DNA, 39 bp, synthetic construct
  misc_feature 1..39: note = Synthetic DNA
  source 1..39: mol_type = other DNA
gcggtggcgatctcccccggctactgtggcactgaggct

SEQ ID NO: 9 - DNA, 39 bp, synthetic construct
  misc_feature 1..39: note = Synthetic DNA
  source 1..39: mol_type = other DNA
ccaggcgatggaagcctcagtgccacagtagccggggga

SEQ ID NO: 10 - DNA, 39 bp, synthetic construct
  misc_feature 1..39: note = Synthetic DNA
  source 1..39: mol_type = other DNA
gtcatcaccatcgacaccatcacctgtctcgaccagccg

SEQ ID NO: 11 - DNA, 39 bp, synthetic construct
  misc_feature 1..39: note = Synthetic DNA
  source 1..39: mol_type = other DNA
tgcccggctgtccggctggtcgagacaggtgatggtgtc

SEQ ID NO: 12 - DNA, 39 bp, synthetic construct
  misc_feature 1..39: note = Synthetic DNA
  source 1..39: mol_type = other DNA
atcagcaaggcctacctggagctgtgtggcgcaacccac

SEQ ID NO: 13 - DNA, 39 bp, synthetic construct
  misc_feature 1..39: note = Synthetic DNA
  source 1..39: mol_type = other DNA
gttcggggcgaagtgggttgcgccacacagctccaggta

SEQ ID NO: 14 - DNA, 39 bp, synthetic construct
  misc_feature 1..39: note = Synthetic DNA
  source 1..39: mol_type = other DNA
cgcgacggactcttcggcgaggtctgtgagtaccgctcc

SEQ ID NO: 15 - DNA, 39 bp, synthetic construct
  misc_feature 1..39: note = Synthetic DNA
  source 1..39: mol_type = other DNA
gaacgggcaggtggagcggtactcacagacctcgccgaa

SEQ ID NO: 16 - DNA, 39 bp, synthetic construct
  misc_feature 1..39: note = Synthetic DNA
  source 1..39: mol_type = other DNA
gcctatctggaactgtgtggtgccacccattttgccccg

SEQ ID NO: 17 - DNA, 39 bp, synthetic construct
  misc_feature 1..39: note = Synthetic DNA
  source 1..39: mol_type = other DNA
cggggcaaaatgggtggcaccacacagttccagataggc

SEQ ID NO: 18 - DNA, 30 bp, synthetic construct
  misc_feature 1..30: note = Synthetic DNA
  source 1..30: mol_type = other DNA
ctgttcggcgaagtgtgtgaataccgcagc

SEQ ID NO: 19 - DNA, 30 bp, synthetic construct
  misc_feature 1..30: note = Synthetic DNA
  source 1..30: mol_type = other DNA
gctgcggtattcacacacttcgccgaacag

SEQ ID NO: 20 - DNA, 24 bp, synthetic construct
  misc_feature 1..24: note = Synthetic DNA
  source 1..24: mol_type = other DNA
gccaacccctgtgagcgcggcccc

SEQ ID NO: 21 - DNA, 24 bp, synthetic construct
  misc_feature 1..24: note = Synthetic DNA
  source 1..24: mol_type = other DNA
ggggccgcgctcacaggggttggc

SEQ ID NO: 22 - DNA, 29 bp, synthetic construct
  misc_feature 1..29: note = Synthetic DNA
  source 1..29: mol_type = other DNA
tcaagcggttcgtctgtaacgacacccgc

SEQ ID NO: 23 - DNA, 29 bp, synthetic construct
  misc_feature 1..29: note = Synthetic DNA
  source 1..29: mol_type = other DNA
tagcgggtgtcgttacagacgaaccgctt

SEQ ID NO: 24 - DNA, 30 bp, synthetic construct
  misc_feature 1..30: note = Synthetic DNA
  source 1..30: mol_type = other DNA
aacccctacgagtgtggccccaacccgacc

SEQ ID NO: 25 - DNA, 30 bp, synthetic construct
  misc_feature 1..30: note = Synthetic DNA
  source 1..30: mol_type = other DNA
cgggttggggccacactcgtaggggttggc

SEQ ID NO: 26 - DNA, 29 bp, synthetic construct
  misc_feature 1..29: note = Synthetic DNA
  source 1..29: mol_type = other DNA
gcatcgcctcccactgtttcgtcgtcatc

SEQ ID NO: 27 - DNA, 28 bp, synthetic construct
  misc_feature 1..28: note = Synthetic DNA
  source 1..28: mol_type = other DNA
tgatgacgacgaaacagtgggaggcgat

SEQ ID NO: 28 - DNA, 28 bp, synthetic construct
  misc_feature 1..28: note = Synthetic DNA
  source 1..28: mol_type = other DNA
cgacgccctgctctgtgccagcagcggc

SEQ ID NO: 29 - DNA, 28 bp, synthetic construct
  misc_feature 1..28: note = Synthetic DNA
  source 1..28: mol_type = other DNA
ggccgctgctggcacagagcagggcgtc

SEQ ID NO: 30 - DNA, 28 bp, synthetic construct
  misc_feature 1..28: note = Synthetic DNA
  source 1..28: mol_type = other DNA
gaacatccccaactgtatcatcggcaag

SEQ ID NO: 31 - DNA, 28 bp, synthetic construct
  misc_feature 1..28: note = Synthetic DNA
  source 1..28: mol_type = other DNA
acttgccgatgatacagttggggatgtt

SEQ ID NO: 32 - DNA, 26 bp, synthetic construct
  misc_feature 1..26: note = Synthetic DNA
  source 1..26: mol_type = other DNA
agcgccagcggctgtggcggcggcac

SEQ ID NO: 33 - DNA, 27 bp, synthetic construct
  misc_feature 1..27: note = Synthetic DNA
  source 1..27: mol_type = other DNA
ggtgccgccgccacagccgctggcgct

SEQ ID NO: 34 - DNA, 28 bp, synthetic construct
  misc_feature 1..28: note = Synthetic DNA
  source 1..28: mol_type = other DNA
cagccgggcagagtgtctcaacgccgcg

SEQ ID NO: 35 - DNA, 28 bp, synthetic construct
  misc_feature 1..28: note = Synthetic DNA
  source 1..28: mol_type = other DNA
gcgcggcgttgagacactctgcccggct

SEQ ID NO: 36 - DNA, 30 bp, synthetic construct
  misc_feature 1..30: note = Synthetic DNA
  source 1..30: mol_type = other DNA
ccagcggcttcggctgtggcaccatctact

SEQ ID NO: 37 - DNA, 30 bp, synthetic construct
  misc_feature 1..30: note = Synthetic DNA
  source 1..30: mol_type = other DNA
agtagatggtgccacagccgaagccgctgg

SEQ ID NO: 38 - DNA, 30 bp, synthetic construct
  misc_feature 1..30: note = Synthetic DNA
  source 1..30: mol_type = other DNA
gagcagctcaactgtgcgctgaaccacatg

SEQ ID NO: 39 - DNA, 30 bp, synthetic construct
  misc_feature 1..30: note = Synthetic DNA
  source 1..30: mol_type = other DNA
catgtggttcagcgcacagttgagctgctc

SEQ ID NO: 40 - DNA, 27 bp, synthetic construct
  misc_feature 1..27: note = Synthetic DNA
  source 1..27: mol_type = other DNA
cgtcccgacctgtgtgccgccatcccg

SEQ ID NO: 41 - DNA, 26 bp, synthetic construct
  misc_feature 1..26: note = Synthetic DNA
  source 1..26: mol_type = other DNA
gggatggcggcacacaggtcgggacg

SEQ ID NO: 42 - DNA, 30 bp, synthetic construct
  misc_feature 1..30: note = Synthetic DNA
  source 1..30: mol_type = other DNA
ggctcaagcggttctgtgacaacgacaccc

SEQ ID NO: 43 - DNA, 30 bp, synthetic construct
  misc_feature 1..30: note = Synthetic DNA
  source 1..30: mol_type = other DNA
gggtgtcgttgtcacagaaccgcttgagcc

SEQ ID NO: 44 - DNA, 27 bp, synthetic construct
  misc_feature 1..27: note = Synthetic DNA
  source 1..27: mol_type = other DNA
gccgccatcccgtgtaccccgtggcac

SEQ ID NO: 45 - DNA, 27 bp, synthetic construct
  misc_feature 1..27: note = Synthetic DNA
  source 1..27: mol_type = other DNA
gtgccacggggtacacgggatggcggc

SEQ ID NO: 46 - DNA, 26 bp, synthetic construct
  misc_feature 1..26: note = Synthetic DNA
  source 1..26: mol_type = other DNA
cccacttcgcctgtaacatccccaac

SEQ ID NO: 47 - DNA, 27 bp, synthetic construct
  misc_feature 1..27: note = Synthetic DNA
  source 1..27: mol_type = other DNA
gttggggatgttacaggcgaagtgggt

SEQ ID NO: 48 - DNA, 29 bp, synthetic construct
  misc_feature 1..29: note = Synthetic DNA
  source 1..29: mol_type = other DNA
gcagcgtcaccgtgtgtacgctgatcatc

SEQ ID NO: 49 - DNA, 28 bp, synthetic construct
  misc_feature 1..28: note = Synthetic DNA
  source 1..28: mol_type = other DNA
cgatgatcagcgtacacacggtgacgct

SEQ ID NO: 50 - DNA, 28 bp, synthetic construct
  misc_feature 1..28: note = Synthetic DNA
  source 1..28: mol_type = other DNA
gccgagctccatctgtaaggcctacctg

SEQ ID NO: 51 - DNA, 28 bp, synthetic construct
  misc_feature 1..28: note = Synthetic DNA
  source 1..28: mol_type = other DNA
ccaggtaggccttacagatggagctcgg

SEQ ID NO: 52 - DNA, 28 bp, synthetic construct
  misc_feature 1..28: note = Synthetic DNA
  source 1..28: mol_type = other DNA
catcgggtgtgacctcgacacgatcgcg

SEQ ID NO: 53 - DNA, 28 bp, synthetic construct
  misc_feature 1..28: note = Synthetic DNA
  source 1..28: mol_type = other DNA
cgaggtcacacccgatgatcagcgtcgg

SEQ ID NO: 54 - DNA, 27 bp, synthetic construct
  misc_feature 1..27: note = Synthetic DNA
  source 1..27: mol_type = other DNA
gcgcaacccacttctgtccgaacatcc

SEQ ID NO: 55 - DNA, 26 bp, synthetic construct
  misc_feature 1..26: note = Synthetic DNA
  source 1..26: mol_type = other DNA
ggggatgttcggacagaagtgggttg